(19) United States
(12) Patent Application Publication
(10) Pub. No.: US 2019/0029210 A1
(43) Pub. Date: Jan. 31, 2019

(54) Spinach Variety NUN 06212 SPS

(71) Applicant: NUNHEMS B.V., Nunhem (NL)

(72) Inventor: Philip SIMONS, Haelen (NL)

(21) Appl. No.: 16/150,037

(22) Filed: Oct. 2, 2018

(51) Int. Cl. A01H 6/02; A01H 5/12

(52) U.S. Cl. CPC A01H 6/028; A01H 5/12

(57) ABSTRACT

The disclosure provides a new and distinct hybrid variety of spinach, NUN 06212 SPS as well as seeds and plants and leaves thereof.

### FIELD OF THE DISCLOSURE

The present disclosure relates to the field of plant breeding and, more specifically, to spinach variety NUN 06212 SPS. The disclosure further relates to vegetative reproductions of NUN 06212 SPS, methods for tissue culture of NUN 06212 SPS and regenerating a plant from such a tissue culture, and to phenotypic variants of NUN 06212 SPS.

### BACKGROUND

The goal of vegetable breeding is to combine various desirable traits in a single variety. Such desirable traits may include greater yield, resistance to insects or pests, tolerance to heat and drought, better agronomic quality, higher nutritional value, growth rate and leaf properties.

Breeding techniques take advantage of a plant's method of pollination. There are two general methods of pollination: self-pollination and cross-pollination.

Plants that have been self-pollinated and selected for type over many generations become homozygous at almost all gene loci and produce a uniform population of true breeding progeny, a homozygous plant. A cross between two such homozygous plants of different varieties produces a uniform population of hybrid plants that are heterozygous for many gene loci. Conversely, a cross of two plants each heterozygous at a number of loci produces a population of hybrid plants that differ genetically and are not uniform. The resulting non-uniformity makes performance unpredictable.

The development of uniform varieties requires the development of homozygous inbred plants, the crossing of these inbred plants, and the evaluation of the crosses. Pedigree breeding and recurrent selection are examples of breeding methods that have been used to develop inbred plants from breeding populations. Those breeding methods combine the genetic backgrounds from two or more plants or various other broad-based sources into breeding pools from which new lines are developed by selfing and selection of desired phenotypes. The new lines are evaluated to determine which of those have commercial potential.

Spinach (Spinacia oleracea) is a flowering plant in the family Amaranthaceae. Spinach is an annual plant (rarely biennial) having flowers that mature into a small hard dry lumpy fruit cluster about 5-10 mm across containing several seeds.

Spinach has two stages in its life cycle including the vegetative, rosette stage in which the plant is marketable (about 35-40 days) and the bolting, seed stalk stage in which the plant is no longer marketable. Spinach can grow in a range of soils as long as they are moist and fertile, and particularly sandy loams that are high in organic matter.

While breeding efforts to date have provided a number of useful spinach lines with beneficial traits, there remains a need in the art for new varieties with further improved traits. Such plants would benefit farmers and consumers alike by improving crop yields and/or quality.

### SUMMARY OF THE VARIOUS ASPECTS OF THE DISCLOSURE

The disclosure provides for spinach variety NUN 06212 SPS, products thereof, and methods of using the same. NUN 06212 SPS is a fresh market spinach and suitable for use as baby-leaf spinach in the field for both conventional and organic production.

In one aspect, the disclosure provides a seed of spinach variety NUN 06212 SPS, wherein a representative sample of said seed has been deposited under Accession Number NCIMB 43217. The disclosure also provides for a plurality of seeds of NUN 06212 SPS. The spinach seed of NUN 06212 SPS may be provided as an essentially homogeneous population of spinach seed. The population of seed of NUN 06212 SPS may be particularly defined as being essentially free from other seed. The seed population may be grown into plants to provide an essentially homogeneous population of spinach plants as described herein.

The disclosure also provides for a plant grown from a seed of spinach variety NUN 06212 SPS and a plant part thereof. In another aspect, the disclosure provides for a hybrid variety of NUN 06212 SPS. The disclosure also provides for a progeny of NUN 06212 SPS. In another aspect, the disclosure provides a plant or a progeny retaining all or all but one, two or three of the “distinguishing characteristics” or all but one, two, three of the “morphological and physiological characteristics” of NUN 06212 SPS, and methods of producing that plant or progeny.

In one aspect, the disclosure provides a plant or a progeny having all the physiological and morphological characteristics of variety NUN 06212 SPS when grown under the same environmental conditions. In another aspect, the plant or progeny has all or all but one, two or three of the physiological and morphological characteristics of NUN 06212 SPS when measured under the same environmental conditions and e.g., evaluated at significance levels of 1%, 5% or 10% significance (which can also be expressed as a p-value) for quantitative characteristics, wherein a representative sample of seed of variety NUN 06212 SPS has been deposited under Accession Number NCIMB 43217. In another aspect, the plant or a progeny has all or all but one, two or three of the physiological and morphological characteristics as listed in Table 1 for variety NUN 06212 SPS when measured under the same environmental conditions and e.g., evaluated at significance levels of 1%, 5% or 10% significance (which can also be expressed as a p-value) for quantitative characteristics.

In another aspect, a plant of NUN 06212 SPS or a progeny thereof has 6, 7, or all of the following distinguishing characteristics of Table 1: 1) leaf blade intensity of green color; 2) leaf blade blistering; 3) leaf blade lobing; 4) leaf petiole attitude; 5) leaf petiole length; 6) leaf blade attitude; 7) leaf blade shape; and 8) cotyledon length.

In another aspect, the spinach plant of NUN 06212 SPS or progeny thereof comprises the RPF11 gene, conferring resistance against at least Peronospora farinosa races 7-14 (preferably against races 7-15; more preferably against races 1, 2, 3, 5, 6 and 7-15), as described in U.S. Pat. No. 9,624,507 and US2017/0027126, both hereby incorporated by reference in their entirety.

In another aspect, the disclosure provides for a plant part obtained from variety NUN 06212 SPS, wherein said plant part is selected from the group consisting of: a leaf, a harvested leaf, a part of a leaf, a fruit, a part of a fruit, pollen, an ovule, a cell, a petiole, a shoot or a part thereof, a stem or a part thereof, a root or a part thereof, a root tip, a cutting, a seed, a part of a seed, seed coat or another maternal tissue which is part of a seed grown on said varieties, hypocotyl, cotyledon, a scion, a stock, a rootstock, a pistil, an anther, and a flower or a part thereof. Leaves are particularly important plant parts. In another aspect, the plant part obtained from variety NUN 06212 SPS is a cell, optionally a cell in a cell or tissue culture. That cell may be grown into a plant of NUN 06212 SPS.

The disclosure also provides a cell culture of NUN 06212 SPS and a plant regenerated from NUN 06212 SPS, wherein the plant has all the characteristics of NUN 06212 SPS when grown under the same environmental conditions, as well as methods for regenerating NUN 06212 SPS. Alternatively, a regenerated plant may have one characteristic that is different from NUN 06212 SPS.

The disclosure further provides a vegetatively propagated plant of variety NUN 06212 SPS having all or all but one, two or three of the morphological and physiological characteristics NUN 06212 SPS when grown under the same environmental conditions.

The disclosure also provides a spinach leaf produced on a plant grown from a seed of NUN 06212 SPS.

In another aspect, the disclosure provides a seed growing or grown on a plant of NUN 06212 SPS (e.g., produced after pollination of the flower of NUN 06212 SPS).

### Definitions

“Spinach” refers herein to plants of the species Spinacia oleracea and leaves thereof. The most commonly eaten part of a spinach is the leaf.

“Cultivated spinach” refers to plants of Spinacia oleracea L (e.g., varieties, breeding lines or cultivars of the species Spinacia oleracea L, cultivated by humans) and having good agronomic characteristics.

The terms “Spinach plant designated NUN 06212 SPS”, “NUN 06212 SPS”, “NUN 06212”, “NUN 06212 F1”, “06212 SPS” or “Spinach 06212” are used interchangeably herein and refer to a spinach plant of variety NUN 06212 SPS, representative seed of which has been deposited under Accession Number NCIMB 43217.

A “seed of NUN 06212 SPS” refers to a spinach seed which can be grown into a plant of NUN 06212 SPS, wherein a representative sample of viable seed of NUN 06212 SPS has been deposited under Accession Number NCIMB 43217. A seed can be in any stage of maturity, for example, a mature, viable seed, or an immature, non-viable seed. A seed comprises an embryo and maternal tissues.

An “embryo of NUN 06212 SPS” refers to an “F1 hybrid embryo” as present in a seed of NUN 06212 SPS, a representative sample of said seed of NUN 06212 SPS has been deposited under Accession Number NCIMB 43217.

A “seed grown on NUN 06212 SPS” refers to a seed grown on a mature plant of NUN 06212 SPS or inside a fruit of NUN 06212 SPS. The “seed grown on NUN 06212 SPS” contains tissues and DNA of the maternal parent, NUN 06212 SPS. The “seed grown on NUN 06212 SPS” contains an F1 embryo. When said seed is planted, it grows into a first generation progeny plant of NUN 06212 SPS.

An “essentially homogeneous population of spinach seed” is a population of seeds where at least 97%, 98%, 99% or more of the total population of seed are seed of NUN 06212 SPS.

An “essentially homogeneous population of spinach plants” is a population of plants where at least 97%, 98%, 99% or more of the total population of plants are plants of NUN 06212 SPS.

The phrase “essentially free from other seed” refers to a population of seed where less than 3%, 2% 1% or even less, of the total population of seed is seed that is not a spinach seed, or in another option, less than 3%, 2% 1%, or less, of the total population of seeds is seed that is not seed of NUN 06212 SPS.

“Tissue culture” or “cell culture” refers to a composition comprising isolated cells of the same or a different type or a collection of such cells organized into parts of a plant. Tissue culture of various tissues of cucumber and regeneration of plants therefrom is well known and widely published (see, e.g., Ren et al., In Vitro Cell.Dev.Biol.—Plant (2013) 49:223-229; Colijn-Hooymans (1994), Plant Cell, Tissue and Organ Culture 39: 211-217). Similarly, the skilled person is well-aware how to prepare a “tissue culture” or “cell culture”.

“USDA descriptors” are the plant variety descriptors described for spinach in the “Objective description of Variety Spinach Spinacia oleracea L.)”, as published by the US Department of Agriculture, Agricultural Marketing Service, Plant Variety Protection Office, Beltsville, Md. 20705, and which can be downloaded from the world-wide web at ams.usda.gov/under services/plant-variety-protection/pvpo-c-forms under spinach. “Non-USDA descriptors” are descriptors suitable for describing spinach.

“UPOV descriptors” are the plant variety descriptors described for spinach in the “Guidelines for the Conduct of Tests for Distinctness, Uniformity and Stability, TG55/7 (Geneva 2007, last updated 2016 Mar. 16), as published by UPOV (International Union for the Protection of New Varieties and Plants,) and which can be downloaded from the world-wide web at upov.int/under edocs/tgdocs/en/tg055.pdf, and is herein incorporated by reference in its entirety. Likewise, “UPOV methods” to determine specific parameters for the characterization of lettuce are described at upov.int.

“RHS” or “RHS color” refers to the color chart of the Royal Horticultural Society (UK) which publishes a botanical color chart quantitatively identifying colors by a defined numbering system. The chart may be purchased from Royal Horticulture Society Enterprise Ltd RHS Garden; Wisley, Woking; Surrey GU236QB, UK, e.g., the RHS color chart 2007.

“Plant part” includes any part of the plant, such as a plant organ (e.g., harvested or non-harvested leaves), a plant cell, a plant protoplast, a plant cell tissue culture or a tissue culture from which a whole plant can be regenerated, a plant cell that is intact in a plant, a clone, a micropropagation, plant callus, a plant cell clump, a plant transplant, a vegetative propagation, a seedling, a fruit, a harvested fruit, a part of a fruit, a leaf, a part of a leaf, pollen, an ovule, an embryo, a petiole, a shoot or a part thereof, a stem or a part thereof, a root or a part thereof, a root tip, a cutting, a seed, a part of a seed, hypocotyl, cotyledon, a scion, a graft, a stock, a rootstock, a pistil, an anther, and a flower or parts of any of these and the like. Also, any developmental stage is included, such as seedlings, cuttings prior or after rooting, mature plants or leaves. Alternatively, a plant part may also include a plant seed which comprises one or two sets of chromosomes derived from the parent plant, e.g., from NUN 06212 SPS. An F1 progeny produced from self-pollination of NUN 06212 SPS will thus comprise two sets of chromosomes derived from NUN 06212 SPS, while an F1 progeny derived from cross-fertilization of NUN 06212 SPS will comprise only one set of chromosomes from NUN 06212 SPS and the other set of chromosomes from the other parent.

“Reference Variety” refers herein to variety NUN 02179 SPS, a variety from Nunhems B. V., with commercial name Antalia. UPOV descriptors of NUN 06212 SPS were compared to the UPOV descriptors and other characteristics of Antalia.

“Harvested plant material” refers herein to a plant part (e.g., a leaf detached from the whole plant) which have been collected for further storage and/or further use.

“Harvest maturity” is referred to as the stage at which a spinach leaf is ready for harvest or the optimal time to harvest the fruit for the market, for processing or for consumption. Spinach leaves may also be harvested at the “baby leaf” stage.

“Yield” means the total weight of all spinach leaves harvested per hectare of a particular line or variety. It is understood that “yield” expressed as weight of all Spinach leaves harvested per hectare can be obtained by multiplying the number of plants per hectare times the “yield per plant”. “Marketable yield” means the total weight of all marketable spinach leaves, especially leaves that is not damaged or diseased, harvested per hectare of a particular line or variety.

A plant having “all the physiological and morphological characteristics” of a referred-to-plant means a plant showing the physiological and morphological characteristics of the referred-to-plant when grown under the same environmental conditions, preferably in the same experiment; the referred-to-plant can be a plant from which it was derived, e.g., the progeny plant, the progenitor plant, the parent, the recurrent parent, the plant used for tissue- or cell culture, etc. A physiological or morphological characteristic can be a numerical characteristic or a non-numerical characteristic. In one aspect, a plant has “all but one, two or three of the physiological and morphological characteristics” of a referred-to-plant, or “all the physiological and morphological characteristics” of Table 1 or “all or all but one, two or three of the physiological and morphological characteristics” of Table 1.

The physiological and/or morphological characteristics mentioned above are commonly evaluated at significance levels of 1%, 5% or 10% if they are numerical (quantitative), or for having an identical degree (or type) if not numerical (not quantitative), if measured under the same environmental conditions. For example, a progeny plant or a Single Locus Converted plant or a mutated plant of NUN 06212 SPS may have one or more (or all) of the essential physiological and/or morphological characteristics of said variety listed in Table 1 when grown under the same environmental conditions.

“Distinguishing characteristics” or “distinguishing morphological and/or physiological characteristics” refers herein to the characteristics which distinguish between the new variety and other spinach varieties, such as the Reference Variety (i.e., are different), when grown under the same environmental conditions. The distinguishing characteristics between NUN 06212 SPS and Reference Variety are described herein and can be seen in Table 1. When comparing NUN 06212 SPS to other varieties, the distinguishing characteristics may be different. In one aspect, the distinguishing characteristics may therefore include at least one, two, three or more (or all) of the characteristics listed in Table 1. All numerical distinguishing characteristics are statistically significantly different at p<0.05 between NUN 06212 SPS and the other variety (e.g., Reference Variety).

NUN 06212 SPS has the following distinguishing characteristics as listed in Table 1 when compared to the Reference Variety: 1) leaf blade intensity of green color; 2) leaf blade blistering; 3) leaf blade lobing; 4) leaf petiole attitude; 5) leaf petiole length; 6) leaf blade attitude; 7) leaf blade shape; and 8) cotyledon length. This can be seen, for example, in Table 1, where the UPOV characteristics of NUN 06212 SPS are compared to the characteristics of Reference Variety, when grown under the same environmental conditions.

Thus, a spinach plant “comprising the distinguishing characteristics of NUN 06212 SPS” (such as a progeny plant) refers herein to a plant which does not differ significantly from said variety in the distinguishing characteristics above. Therefore, in one aspect, the disclosure provides a plant that does not differ significantly from NUN 06212 SPS in the distinguishing characteristics above.

Similarity and differences between two different plant lines or varieties can be determined by comparing the number of morphological and/or physiological characteristics (e.g., the characteristics as listed in Table 1) that are the same (i.e., statistically not significantly different) or that are different (i.e., statistically significantly different) between the two plant lines or varieties when grown under the same environmental conditions. A numerical characteristic is considered to be “the same” when the value for a numeric characteristic is not significantly different at the 1% (p<0.01) or 5% (p<0.05) significance level, using one-way Analysis of variance (ANOVA), a standard method known to the skilled person. Non-numerical or “degree” or “type” characteristic is considered “the same” when the values have the same “degree” or “type” when scored using USDA and/or UPOV descriptors, if the plants are grown under the same environmental conditions.

As used herein, the term “variety”, “cultivated spinach” or “cultivar” means a plant grouping within a single botanical taxon of the lowest known rank, which grouping, irrespective of whether the conditions for the grant of a breeder's right are fully met, can be defined by the expression of the characteristics resulting from a given genotype or combination of genotypes, distinguished from any other plant grouping by the expression of at least one of the said characteristics and considered as a unit with regard to its suitability for being propagated unchanged.

A “plant line” is for example a breeding line which can be used to develop one or more varieties. A breeding line is typically highly homozygous.

“Hybrid variety” or “F1 hybrid” refers to the seeds harvested from crossing two inbred (nearly homozygous) parental lines. For example, the female parent is pollinated with pollen of the male parent to produce hybrid (F1) seeds on the female parent.

“Regeneration” refers to the development of a plant from cell culture or tissue culture or vegetative propagation.

“Vegetative propagation”, “vegetative reproduction” or “clonal propagation” are used interchangeably herein and mean a method of taking a part of a plant and allowing that plant part to form at least roots, and also refer to the plant or plantlet obtained by that method. Optionally, the vegetative propagation is grown into a mature plant. The skilled person is aware of what plant parts are suitable for use in the method.

“Selfing” refers to self-pollination of a plant, i.e., the transfer of pollen from the anther to the stigma of the same plant.

“Crossing” refers to the mating of two parent plants. The term encompasses “cross-pollination” and “selfing”.

“Cross-pollination” refers to the fertilization by the union of two gametes from different plants.

As used herein, the terms “resistance” and “tolerance” are used interchangeably to describe plants that show no symptoms or significantly reduced symptoms to a specified biotic pest, pathogen, abiotic influence or environmental condition compared to a susceptible plant. These terms are optionally also used to describe plants showing some symptoms but that are still able to produce marketable product with an acceptable yield.

The term “traditional breeding techniques” encompasses herein crossing, selfing, selection, doubled haploid production, embryo rescue, protoplast fusion, marker assisted selection, mutation breeding etc. as known to the breeder (e.g., methods other than genetic modification/transformation/transgenic methods), by which, for example, a genetically heritable trait can be transferred from one spinach line or variety to another.

“Backcrossing” is a traditional breeding technique used to introduce a trait into a plant line or variety. The plant containing the trait is called the donor plant and the plant into which the trait is transferred is called the recurrent parent. An initial cross is made between the donor parent and the recurrent parent to produce a progeny plant. Progeny plants which have the trait are then crossed to the recurrent parent. After several generations of backcrossing and/or selfing the recurrent parent comprises the trait of the donor. The plant generated in this way may be referred to as a “single trait converted plant”. The technique can also be used on a parental line of a hybrid.

“Progeny” as used herein refers to a plant obtained from a plant designated NUN 06212 SPS. A progeny may be obtained by regeneration of cell culture or tissue culture or parts of a plant of said variety or selfing of a plant of said variety or by producing seeds of a plant of said variety. In further aspects, progeny may also encompass plants obtained from crossing of at least one plant of said variety with another spinach plant of the same variety or another variety or (breeding) line, or with wild spinach plants. A progeny may comprise a mutation or a transgene. A “first-generation progeny” or is the progeny directly derived from, obtained from, obtainable from or derivable from the parent plant by, e.g., traditional breeding methods (selfing and/or cross-pollinating) or regeneration (optimally combined with transformation or mutation). Thus, a plant of NUN 06212 SPS is the male parent, the female parent or both of a first-generation progeny of NUN 06212 SPS. Progeny may have all the physiological and morphological characteristics of variety NUN 06212 SPS when grown under the same environmental conditions. Using common breeding methods such as backcrossing or recurrent selection, mutation or transformation, one or more specific characteristics may be introduced into said variety, to provide or a plant comprising all but 1, 2, or 3 or more of the morphological and physiological characteristics of NUN 06212 SPS.

The terms “gene converted” or “conversion plant” or “single locus converted plant” in this context refer to spinach plants which are developed by traditional breeding techniques e.g., backcrossing, or via genetic engineering or through mutation breeding, wherein essentially all of the desired morphological and physiological characteristics of the parent variety or line are recovered, in addition to the one or more characteristics introduced into the parent via e.g., the backcrossing technique (optionally including reverse breeding or reverse synthesis of breeding lines). It is understood that not only the addition of a further characteristic (e.g., addition of gene conferring a further characteristic, such as a disease resistance gene), but also the replacement/modification of an existing characteristic by a different characteristic is encompassed herein (e.g., mutant allele of a gene can modify the phenotype of a characteristic).

Likewise, a “Single Locus Converted (Conversion) Plant” refers to plants which are developed by plant breeding techniques comprising or consisting of mutation breeding and/or by genetic transformation and/or by traditional breeding techniques, such as backcrossing, wherein essentially all of the desired morphological and physiological characteristics of a spinach variety are recovered in addition to the characteristics of the single locus having been transferred into the variety via the above mentioned technique, or wherein a morphological and physiological characteristic of the variety has been replaced/modified in the variety. In case of a hybrid, the gene may be introduced, or modified in the male or female parental line.

“Average” refers herein to the arithmetic mean.

The term “mean” refers to the arithmetic mean of several measurements. The skilled person understands that the appearance of a plant depends to some extent on the growing conditions of said plant. Thus, the skilled person will know typical growing conditions for NUN 06212 SPS. The mean, if not indicated otherwise within this application, refers to the arithmetic mean of measurements on at least 10 different, randomly selected plants of a variety or line.

### DETAILED DESCRIPTION OF VARIOUS ASPECTS OF THE DISCLOSURE

The disclosure relates to a plant of NUN 06212 SPS, wherein a representative sample of seeds of said variety has been deposited under the Budapest Treaty, with Accession number NCIMB 43217. NUN 06212 SPS is a fresh market spinach and suitable for use as baby-leaf spinach in the field for both conventional and organic production.

The disclosure also relates to a seed of spinach variety, referred to as NUN 06212 SPS, wherein a representative sample of said seed has been deposited under the Budapest Treaty, with Accession number NCIMB 43217.

In another aspect, the disclosure provides for a spinach plant part of variety NUN 06212 SPS, preferably a leaf, a representative sample of seed from said variety has been deposited under the Budapest Treaty, with Accession number NCIMB 43217.

A seed of hybrid variety NUN 06212 SPS is obtainable by crossing the male parent of said variety with the female parent of said variety and harvesting the seeds produced on the female parent. The resultant seeds of said variety can be grown to produce plants of said variety. In one aspect, a seed or a plurality of seeds of said variety are packaged into a container of any size or type (e.g., bags, cartons, cans, etc.). The seed may be disinfected, primed and/or treated with various compounds, such as seed coatings or crop protection compounds. The seed produces a plant of NUN 06212 SPS.

Also provided is a plant of spinach variety NUN 06212 SPS, or a leaf or other plant part thereof, produced from a seed, wherein a representative sample of said seeds has been deposited under the Budapest Treaty, with Accession Number NCIMB 43217.

Also provided is a plant part obtained from variety NUN 06212 SPS, wherein said plant part is a leaf, a harvested leaf, a part of a leaf, a fruit, a part of a fruit, pollen, an ovule, a cell, a petiole, a shoot or a part thereof, a stem or a part thereof, a root or a part thereof, a root tip, a cutting, a seed, a part of a seed, seed coat or another maternal tissue which is part of a seed grown on said varieties, hypocotyl, cotyledon, a scion, a stock, a rootstock, a pistil, an anther, and a flower or a part thereof. Leaves are particularly important plant parts. In a further aspect, the plant part obtained from variety NUN 06212 SPS is a cell, optionally a cell in a cell or tissue culture. The cell may be grown into a plant of NUN 06212 SPS. A part of NUN 06212 SPS (or of a progeny that variety or of a plant having all physiological and/or morphological characteristics but one, two or three of NUN 06212 SPS) further encompasses any cells, tissues, organs obtainable from the seedlings or plants in any stage of maturity.

The disclosure also provides for a food or feed product or a processed product comprising or consisting of a plant part described herein. Preferably, the plant part is a spinach leaf or part thereof and/or an extract from a leaf or another plant part described herein comprising at least one cell of NUN 06212 SPS. The food or feed product may be fresh or processed, e.g., dried, grinded, powdered, pickled, chopped, cooked, roasted, in a sauce, in a sandwich, pasted, puréed or concentrated, juiced, pickled, canned, steamed, boiled, fried, blanched and/or frozen, etc.

Such a plant part of NUN 06212 SPS can be stored and/or processed further. The disclosure thus also provides for a food or feed products comprising one or more of such parts, such as canned, chopped, cooked, roasted, in a sauce, in a sandwich, pasted, puréed or concentrated, juiced, frozen, dried, pickled, or powdered spinach fruit from NUN 06212 SPS or from progeny of said variety, or from a derived variety, such as a plant having all but one, two or three physiological and/or morphological characteristics of NUN 06212 SPS.

In another aspect, the disclosure provides for a spinach leaf of variety NUN 06212 SPS, or a part of a leaf thereof of said variety. The leaf can be in any stage of maturity, for example, immature or baby leaf or mature. In another aspect, the disclosure provides for a container comprising or consisting of a plurality of harvested spinach leaves or parts of leaves of said variety, or leaves of progeny thereof, or leaves of a derived variety.

In another aspect, the plant, plant part or seed of NUN 06212 SPS is inside a container, for example, containers such as cans, boxes, crates, bags, cartons, Modified Atmosphere Packaging, films (e.g., biodegradable films), etc. comprising a plant or a part of a plant (fresh and/or processed) or a seed of NUN 06212 SPS. In a particular aspect, the container comprises a plurality of seeds of NUN 06212 SPS, or a plurality of plant parts of NUN 06212 SPS.

The disclosure further relates to a spinach variety, referred to as NUN 06212 SPS, which when compared to its REFERENCE VARIETY (Antalia) has the following distinguishing characteristics of Table 1: 1) leaf blade intensity of green color; 2) leaf blade blistering; 3) leaf blade lobing; 4) leaf petiole attitude; 5) leaf petiole length; 6) leaf blade attitude; 7) leaf blade shape; and 8) cotyledon length, where the characteristics are determined for plants grown under the same environmental conditions. Also encompassed are parts of that plant.

In one aspect, a plant of NUN 06212 SPS or a progeny plant thereof, comprises all of the following morphological and/or physiological characteristics of Table 1: 1) leaf blade intensity of green color; 2) leaf blade blistering; 3) leaf blade lobing; 4) leaf petiole attitude; 5) leaf petiole length; 6) leaf blade attitude; 7) leaf blade shape; and 8) cotyledon length, where the characteristics are determined for plants grown under the same environmental conditions. An example of values for the characteristics collected in a trial run according to UPOV requirements can be found in Table 1. A part of this plant is also provided.

In another aspect, NUN 06212 SPS or progeny thereof comprises resistance to Peronospora farinose races 7-14, or races 7-15, or races 1, 2, 5, 6, and 7-15, as conferred by the RPF11 gene.

Optionally, in one aspect NUN 06212 SPS comprises resistance to Perenospora farinose f. spinacea race 1, 2, 3, 4, 5, 6, 7, 8, 9, 10, 11, 12, 13, 14, 15, 16, and 17, measured according to UPOV standard as described in TG55/7.

The disclosure further provides a spinach plant which does not differ from the plant of NUN 06212 SPS as determined at the 1%, 2%, 3%, 4% or 5% significance level when grown under the same environmental conditions. In a particular aspect, the plants are measured in the same trial (e.g., trial is conducted as recommended by the USDA or UPOV).

The disclosure also provides a tissue or cell culture comprising cells of NUN 06212 SPS. Such a tissue culture can, for example, be grown on plates or in liquid culture, or be frozen for long term storage. The cells of NUN 06212 SPS used to start the culture can be selected from any plant part suitable for vegetative reproduction, or in a particular aspect, can be one or more of an embryo, meristem, cotyledon, hypocotyl, pollen, a leaf, an anther, a root, a root tip, a pistil, a petiole, a flower, a fruit, a seed, a stem or a stalk. In another particular aspect, the tissue culture does not contain somaclonal variation or has reduced somaclonal variation. The skilled person is familiar with methods to reduce or prevent somaclonal variation, including regular reinitiation.

In one aspect, the disclosure provides a spinach plant regenerated from the tissue or cell culture of NUN 06212 SPS, wherein the regenerated plant is not significantly different from NUN 06212 SPS in all, or all but one, two or three, of the physiological and morphological characteristics (e.g., determined at the 5% significance level when grown under the same environmental conditions). Optionally, the plant has one, two or three the physiological and morphological characteristics that are affected by a mutation or by transformation. In another aspect, the disclosure provides a spinach plant regenerated from the tissue or cell culture of NUN 06212 SPS, wherein the plant has all of the physiological and morphological characteristics of said variety determined (e.g., at the 5% significance level) when grown under the same environmental conditions. Similarity or difference of a characteristic is determined by measuring the characteristics of a representative number of plants grown under the same environmental conditions, determining whether type/degree characteristics are the same and determining whether numerical characteristics are different at the 5% significance level.

NUN 06212 SPS, or its progeny, or a plant having all physiological and/or morphological characteristics but one, two or three which are different from those of NUN 06212 SPS, can also be reproduced using vegetative reproduction methods. Therefore, the disclosure provides for a method of producing a plant or a plant part of variety NUN 06212 SPS, comprising vegetative propagation of NUN 06212 SPS. Vegetative propagation comprises regenerating a whole plant from a plant part of NUN 06212 SPS or from a progeny or from a plant having all physiological and/or morphological characteristics of said variety but one, two or three different characteristics), such as a cutting, a cell culture or a tissue culture.

The disclosure also concerns methods of vegetatively propagating a part of the plant of the variety NUN 06212 SPS. In certain aspects, the method comprises the steps of: (a) collecting tissue or cells capable of being propagated from a plant of NUN 06212 SPS to proliferated shoots; and (b) rooting said proliferated shoots, to obtain rooted plantlets. Steps (a) and (b) may also be reversed, i.e., first cultivating said tissue to obtain roots and then cultivating the tissue to obtain shoots, thereby obtaining rooted plantlets. The rooted plantlets may then be further grown, to obtain plants. In one aspect, the method further comprises step (c) growing plants from said rooted plantlets. Therefore, the method also comprises regenerating a whole plant from said part of NUN 06212 SPS. In a particular aspect, the part of the plant to be propagated is a cutting, a cell culture or a tissue culture.

The disclosure also provides for a vegetatively propagated plant of variety NUN 06212 SPS (or from progeny of NUN 06212 SPS or from or a plant having all but one, two or three physiological and/or morphological characteristics of NUN 06212 SPS), wherein the plant has all of the morphological and physiological characteristics of NUN 06212 SPS when the characteristics are determined (e.g., at the 5% significance level) for plants grown under the same conditions. In another aspect, the propagated plant has all but one, two or three of the morphological and physiological characteristics of NUN 06212 SPS when the characteristics are determined (e.g., at the 5% significance level) for plants grown under the same conditions. A part of said propagated plant or said propagated plant with one, two or three differences is also provided.

In another aspect, the disclosure provides a method for producing a plant part, preferably a leaf, comprising growing a plant of NUN 06212 SPS until it develops at least one leaf, and optionally collecting the leaf. Preferably, the leaf is collected at harvest maturity. In another aspect, the leaf is collected at baby leaf stage. A plant of NUN 06212 SPS can be produced by seeding directly in the soil (e.g., field) or by germinating the seeds in controlled environment conditions (e.g., greenhouses, hydroponic cultures, etc.) and optionally then transplanting the seedlings into the field (see, world wide web at //anrcatalog.ucanr.edu/pdf/7216.pdf). For example, the seed can be sown into prepared seed beds where they will remain for the entire production the crop. Spinach can also be grown entirely in greenhouses. Spinach can also be grown in tunnels. Moreover, spinach can be grown in hydroponic cultures as described in, e.g., US2008/0222949, which is herein incorporated by reference in its entirety, and the skilled person is familiar with various types of hydroponics cultures (see, world wide web at cea.cals.cornell.edu/attachments/Cornell CEA baby spinach handbook.pdf).

In still another aspect, the disclosure provides a method of producing a spinach plant, comprising crossing a plant of NUN 06212 SPS with a second spinach plant at least once, allowing seed to develop and optionally harvesting said progeny seed. The skilled person can select progeny from said crossing. Optionally, the progeny is crossed twice, thrice, or four, five, six or seven times, and allowed to set seed. In one aspect, the first “crossing” comprises planting seeds of a first and a second parent spinach plant, often in proximity so that pollination will occur; for example, mediated by insect vectors. Alternatively, pollen can be transferred manually. Where the plant is self-pollinated, pollination may occur without the need for direct human intervention other than plant cultivation. After pollination the plant can produce seed.

In another aspect, the disclosure provides a method of producing a plant, comprising selfing a plant of variety NUN 06212 SPS one or more times, and selecting a progeny plant from said selfing. In one aspect, the progeny plant retains all the distinguishing characteristics of NUN 06212 SPS described above when grown under the same environmental conditions. In a different aspect, the progeny plant comprises all (or all but one, two or three) of the physiological and morphological characteristic of NUN 06212 SPS of Table 1.

In other aspects, the disclosure provides a progeny plant of variety NUN 06212 SPS such as a progeny plant obtained by further breeding of NUN 06212 SPS. Further breeding with NUN 06212 SPS includes selfing that variety and/or cross-pollinating NUN 06212 SPS with another spinach plant one or more times. In particular, the disclosure provides for a progeny plant that retains all the essential morphological and physiological characteristics of NUN 06212 SPS or, in another aspect, a progeny plant that retains all, or all but one, two or three, of the morphological and physiological characteristics of NUN 06212 SPS, optionally all or all but one, two or three of the characteristics as listed in Table 1, when grown under the same environmental conditions. In another aspect, the progeny is a first generation progeny, e.g., the ovule or the pollen (or both) used in the crossing is an ovule or pollen of NUN 06212 SPS, where e.g., the pollen comes from an anther of NUN 06212 SPS and/or the ovule comes from an ovary of NUN 06212 SPS. In another aspect, the disclosure provides for a vegetative reproduction of the variety and a plant having all, or all but 1, 2, or 3 of the physiological and morphological characteristics of NUN 06212 SPS (e.g., as listed in Table 1).

The disclosure also provides a method for collecting pollen of NUN 06212 SPS, comprising collecting pollen from a plant of NUN 06212 SPS. Alternatively, the method comprises growing a plant of NUN 06212 SPS until at least one flower contains pollen and collecting the pollen. In a particular aspect, the pollen is collected when it is mature or ripe. A suitable method for collecting pollen comprises collecting anthers or the part of the anther that contains pollen, for example, by cutting it the anther or the part of the anther off. Pollen can be collected in a container. Optionally, collected pollen can be used to pollinate a spinach flower.

The morphological and/or physiological differences between two different individual plants described herein (e.g., between NUN 06212 SPS and a progeny of NUN 06212 SPS) or between a plant of NUN 06212 SPS or progeny of NUN 06212 SPS, or a plant having all, or all but 1, 2, or 3, of the physiological and morphological characteristics of NUN 06212 SPS (or all, or all but 1, 2, or 3 of the characteristics as listed in Table 1) and another known variety can easily be established by growing said variety next to each other or next to the other variety (e.g., in the same field, under the same environmental conditions), preferably in several locations which are suitable for said spinach cultivation, and measuring morphological and/or physiological characteristics of a number of plants (e.g., to calculate an average value and to determine the variation range/uniformity within the variety). Thus, the disclosure comprises spinach plant having one, two or three physiological and/or morphological characteristics which are different from those of the plant of NUN 06212 SPS, and which otherwise has all the physiological and morphological characteristics of the plant of NUN 06212 SPS, when determined (e.g., at the 5% significance level for quantitative characteristics or determined by type for non-quantitative characteristics) for plants grown under the same environmental conditions. In a particular aspect, the different characteristic(s) is/are a result of breeding with NUN 06212 SPS and selection of a progeny plant comprising 1, 2, or 3 characteristics which are different than in NUN 06212 SPS. In another aspect, the different characteristic is the result of a mutation (e.g., spontaneous mutation or human induced mutation through e.g. targeted mutagenesis or traditional mutagenesis such as chemically or radiation induced mutagenesis), or it is the result of transformation.

The morphological and physiological characteristics (and the distinguishing characteristics) of NUN 06212 SPS are provided, for example, in Table 1. Encompassed herein is also a plant obtainable from NUN 06212 SPS (e.g., by selfing and/or crossing and/or backcrossing with said variety and/or progeny of said variety) comprising all or all but one, two or three of the physiological and morphological characteristics of NUN 06212 SPS listed in Table 1 (as determined at the 5% significance level for numerical characteristics or identical for non-numerical characteristics) when grown under the same environmental conditions and/or comprising one or more (or all; or all except one, two or three) characteristics when grown under the same environmental conditions. The morphological and/or physiological characteristics may vary somewhat with variation in the environment (such as temperature, light intensity, day length, humidity, soil, fertilizer use), which is why a comparison under the same environmental conditions is preferred. Colors can best be measured using the Royal Horticultural Society (RHS) Chart.

In another aspect, the disclosure provides for a method of producing a new spinach plant. The method comprises crossing NUN 06212 SPS, or a plant comprising all but 1, 2, or 3 of the morphological and physiological characteristics of NUN 06212 SPS (as listed in Table 1), or a progeny thereof, either as male or as female parent, with a second spinach plant (or a wild relative of Spinach) one or more times, and/or selfing a spinach plant of NUN 06212 SPS, or a progeny plant thereof, one or more times, and selecting progeny from said crossing and/or selfing. The second spinach plant may, for example, be a line or variety of the species Spinacia oleracea, or Spinacia tetrandra, or Spinacia turkestanica, or other Spinacia species.

The disclosure provides for methods of producing plants which retain all the morphological and physiological characteristics of a plant described herein. The disclosure also provides for methods of producing a plant comprising all but 1, 2, or 3 or more of the morphological and physiological characteristics of NUN 06212 SPS (e.g., as listed in Table 1), but which are still genetically closely related to said variety. The relatedness can, for example, be determined by fingerprinting techniques (e.g., making use of isozyme markers and/or molecular markers such as Single-nucleotide polymorphism (SNP) markers, amplified fragment length polymorphism (AFLP) markers, microsatellites, minisatellites, Random Amplified Polymorphic DNA (RAPD) markers, restriction fragment length polymorphism (RFLP) markers and others). A plant is “closely related” to NUN 06212 SPS if its DNA fingerprint is at least 80%, 90%, 95% or 98% identical to the fingerprint of NUN 06212 SPS. In a particular aspect, AFLP markers are used for DNA fingerprinting (Vos et al. 1995, Nucleic Acid Research 23: 4407-4414). A closely related plant may have a Jaccard's Similarity index of at least about 0.8, preferably at least about 0.9, 0.95, 0.98 or more (Parvathaneni et al., J. Crop Sci. Biotech. 2011 (March) 14 (1): 39-43). The disclosure also provides a plant and a variety obtained or selected by applying these methods on NUN 06212 SPS. Such a plant may be produced by crossing and/or selfing, or alternatively, a plant may simply be identified and selected amongst plants of said variety, or progeny of said variety, e.g., by identifying a variant within NUN 06303 SPS or within progeny of said variety (e.g., produced by selfing) which variant differs from NUN 06212 SPS in one, two or three of the morphological and/or physiological characteristics (e.g., in one, two or three distinguishing characteristics), e.g., those listed in Table 1. In one aspect, the disclosure provides a spinach plant having a Jaccard's Similarity index with NUN 06212 SPS of at least 0.8, e.g., at least 0.85, 0.9, 0.95, 0.98 or even at least 0.99.

The disclosure also provides methods for determining the identity of parental lines of plants described herein, in particular the identity of the female line. US 2015/0126380, which is hereby incorporated by reference, relates to a non-destructive method for analyzing maternal DNA of a seed. In this method, the DNA is dislodged from the seed coat surface and can be used to collect information on the genome of the maternal parent of the seed. This method for analyzing maternal DNA of a seed comprises the steps of contacting a seed with a fluid to dislodge DNA from the seed coat surface, and analyzing the DNA thus dislodged from the seed coat surface using methods known in the art. The skilled person is thus able to determine whether a seed has grown on a plant of a plant of NUN 06212 SPS or is a progeny of said variety, because the seed coat of the seed is a maternal tissue genetically identical to NUN 06212 SPS. In one aspect, the disclosure relates to a seed coat comprising maternal tissue of NUN 06212 SPS. In another particular aspect, the disclosure relates to a spinach seed comprising a maternal tissue of NUN 06212 SPS.

By crossing and/or selfing also (one or more) single traits may be introduced into, or modified in, NUN 06212 SPS (e.g., using backcrossing breeding schemes), while retaining the remaining morphological and physiological characteristics of said variety and/or while retaining one or more or all distinguishing characteristics. A single trait converted plant may thereby be produced. For example, disease resistance genes may be introduced, genes responsible for one or more quality traits, yield, etc. Both single genes (e.g., dominant or recessive) and one or more QTLs (quantitative trait loci) may be transferred into NUN 06212 SPS by breeding with said variety.

Any pest or disease resistance genes may be introduced NUN 06212 SPS, progeny of NUN 06212 SPS or into a plant comprising all but 1, 2, or 3 or more of the morphological and physiological characteristics of NUN 06212 SPS (e.g., as listed in Table 1). Resistance to one or more of the following diseases or pests may be introduced into plants described herein: Peronospora farinosa f.sp. spinaciae, e.g., to race 1-17 or new races and/or other isolates; white rust (Albugo occidentalis), Fusarium oxysporum f.sp. spinaciae, Pythium resistance, Rhizoctonia resistance, Colletotrichum anthracnose resistance, Cercospora beticola resistance, Verticillium dahliae resistance, Phytophthora ssp resistance, Stemphylium leaf spot resistance, Curly Top Virre resistance, Cucumber Mosaic Virus (CMV) resistance, Impatiens Necrotic Spot Virus (INSV), Beet Yellows and/or Beet mosaic resistance, and/or leaf miner resistance. Other resistance genes, against pathogenic viruses, fungi, bacteria, nematodes, insects or other pests may also be introduced.

The disclosure also provides a method for developing a spinach plant in a spinach breeding program, using a spinach plant described herein, or its parts as a source of plant breeding material. Suitable plant breeding techniques are recurrent selection, backcrossing, pedigree breeding, mass selection, mutation breeding and/or genetic marker enhanced selection. In one aspect, the method comprises crossing NUN 06212 SPS or progeny of said variety, or a plant comprising all but 1, 2, or 3 or more of the morphological and physiological characteristics of NUN 06212 SPS (e.g., as listed in Table 1), with a different spinach plant, and wherein one or more offspring of the crossing are subject to one or more plant breeding techniques selected from the group consisting of recurrent selection, backcrossing, pedigree breeding, mass selection, mutation breeding and genetic marker enhanced selection (see e.g., Brotman et al., Theor Appl Genet (2002) 104:1055-1063). For breeding methods in general, see Principles of Plant Genetics and Breeding, 2007, George Acquaah, Blackwell Publishing, ISBN-13: 978-1-4051-3646-4.

The disclosure also provides a spinach plant comprising at least a first set of the chromosomes of spinach variety NUN 06212 SPS, a sample of seed of said variety has been deposited under Accession Number NCIMB 43217; optionally further comprising a single locus conversion or a mutation, wherein said plant has essentially all of the morphological and physiological characteristics of the plant comprising at least a first set of the chromosomes of said variety. In another aspect, this single locus conversion confers a trait selected from the group consisting of male sterility, herbicide tolerance, insect resistance, pest resistance, disease resistance, environmental stress tolerance, Peronospora resistance modified carbohydrate metabolism and modified protein metabolism.

In one aspect, a plant of NUN 06212 SPS may also be mutated (by e.g., irradiation, chemical mutagenesis, heat treatment, etc.) and mutated seeds or plants may be selected in order to change one or more characteristics of said variety. Methods such as TILLING may be applied to spinach populations in order to identify mutants. Similarly, NUN 06212 SPS may be transformed and regenerated, whereby one or more chimeric genes are introduced into the variety or into a plant comprising all but 1, 2, 3, or more of the morphological and physiological characteristics (e.g., as listed in Table 1). Transformation can be carried out using standard methods, such as Agrobacterium tumefaciens mediated transformation or biolistics, followed by selection of the transformed cells and regeneration into plants. A desired trait (e.g., gene(s) conferring pest or disease resistance, herbicide, fungicide or insecticide tolerance, etc.) can be introduced into NUN 06212 SPS, or progeny of said variety, by transforming said variety or progeny of said variety with a transgene that confers the desired trait, wherein the transformed plant retains all or all but one, two or three of the phenotypic and/or morphological and/or physiological characteristics of NUN 06212 SPS or the progeny of said variety and contains the desired trait.

The disclosure also provides a plant or a cell of a plant comprising a desired trait produced by mutating a plant of variety NUN 06212 SPS or a cell thereof and selecting a plant the desired trait, wherein the mutated plant retains all or all but one of the phenotypic and morphological characteristics of said variety, optionally as described for each variety in Table 1, and contains the desired trait and wherein a representative sample of seed of variety NUN 06212 SPS is deposited under Accession Number NCIMB 43217. In a further aspect, the desired trait is selected from the group consisting of yield, storage properties, color, flavor, male sterility, herbicide tolerance, insect resistance, pest resistance, disease resistance, environmental stress tolerance, Peronospora resistance, modified carbohydrate metabolism and modified protein metabolism.

In one aspect, the disclosure provides a method for inducing mutation in NUN 06212 SPS comprising:
a) exposing a seed, a plant or a plant part or a cell of NUN 06212 SPS to a mutagenic chemical or to radiation, wherein a representative sample of seed of NUN 06212 SPS is deposited under Accession Number NCIMB 43217;
b) selecting a seed, a plant or a plant part or a cell of NUN 06212 SPS having a mutation; and
c) optionally growing and/or multiplying the seed, plant or plant part or cell of NUN 06212 SPS having the mutation.

The disclosure also provides a plant having one, two or three physiological and/or morphological characteristics which are different from those of NUN 06212 SPS, and which otherwise has all the physiological and morphological characteristics of said variety, wherein a representative sample of seed of variety NUN 06212 SPS is deposited under Accession Number NCIMB 43217. In particular, variants which differ from NUN 063212 SPS in none, one, two or three of the characteristics mentioned in Table 1 are encompassed.

A part of NUN 06212 SPS (or of progeny of said variety or of a plant having all physiological and/or morphological characteristics but one, two or three which are different from those of said variety) encompasses any cells, tissues, organs obtainable from the seedlings or plants, such as but not limited to: a spinach leaf or a part thereof, a cutting, hypocotyl, cotyledon, seed coat, pollen and the like. Such parts can be stored and/or processed further. The disclosure further provides for food or feed products comprising a part of NUN 06212 SPS or a part of progeny of NUN 06212 SPS, or a part of a plant having all but one, two or three physiological and/or morphological characteristics of NUN 06212 SPS, comprising one or more of such parts, optionally processed (such as canned, chopped, cooked, roasted, in a sauce, in a sandwich, pasted, puréed or concentrated, juiced, frozen, dried, pickled, or powdered).

In one aspect, the disclosure provides for a haploid plant and/or a doubled haploid plant of NUN 06212 SPS, or of a plant having all but one, two or three physiological and/or morphological characteristics of NUN 06212 SPS, or progeny of any of these, is encompassed herein. Haploid and doubled haploid (DH) plants can, for example, be produced by cell or tissue culture and chromosome doubling agents and regeneration into a whole plant. DH production chromosome doubling may be induced using known methods, such as colchicine treatment or the like. In one aspect, the method comprises inducing a cell or tissue culture with a chromosome doubling agent, and regenerating the cells or tissues into a whole plant.

In another aspect, the disclosure comprises a method for making doubled haploid cells from haploid cells of NUN 06212 SPS is comprising doubling cells of NUN 06212 SPS with a chromosome doubling agent, such as colchicine treatment (Nikolova and Niemirowicz-Szczytt (1996) Acta Soc Bot Pol 65:311-317).

In any of the above methods, wherein the single locus conversion concerns a trait, the trait may be yield or pest resistance or disease resistance. In one aspect, the trait is disease resistance and the resistance is conferred to Peronospora farinosa f.sp. spinaciae, e.g., to race 1-17 or new races and/or other isolates; white rust (Albugo occidentalis), Fusarium oxysporum f.sp. spinaciae, Pythium resistance, Rhizoctonia resistance, Colletotrichum anthracnose resistance, Cercospora beticola resistance, Verticillium dahliae resistance, Phytophthora ssp resistance, Stemphylium leaf spot resistance, Curly Top Virus resistance, Cucumber Mosaic Virus (CMV) resistance, Impatiens Necrotic Spot Virus (INSV), Beet Yellows and/or Beet mosaic resistance, or leaf miner resistance.

The disclosure also provides a combination of parental lines which, when crossed, produce a seed or plant having all physiological and/or morphological characteristics of NUN 06212 SPS but one, two or three which are different (when grown under the same environmental conditions), as well as a seed or plant having all physiological and/or morphological characteristics of NUN 06212 SPS but one, two or three which are different (e.g., when the characteristics are determined at the 5% significance level for plants grown under the same conditions).

Also provided is a plant part obtainable from variety NUN 06212 SPS or from progeny of said variety or from a plant having all but one, two or three physiological and/or morphological characteristics which are different from those of NUN 06212 SPS, or from a vegetatively propagated plant of NUN 06212 SPS (or from its progeny or from a plant having all or all but one, two or three physiological and/or morphological characteristics which are different from those of NUN 06212 SPS), being selected from the group consisting of a leaf, a part of a leaf, a harvested leaf, a fruit, a part of a fruit, pollen, an ovule, a cell, a petiole, a shoot or a part thereof, a stem or a part thereof, a root or a part thereof, a root tip, a cutting, a seed, a part of a seed, seed-coat or another maternal tissue which is part of a seed grown on NUN 06212 SPS, or hypocotyl, cotyledon, a scion, a stock, a rootstock, a pistil, an anther, and a flower or a part thereof.

In another aspect, the disclosure provides a method of determining the genotype of a plant described herein comprising detecting in the genome (e.g., a sample of nucleic acids) of the plant at least a first polymorphism or an allele. The skilled person is familiar with many suitable methods of genotyping, detecting a polymorphism or detecting an allele including restriction fragment length polymorphism identification (RFLP) of genomic DNA, random amplified polymorphic detection (RAPD) of genomic DNA, amplified fragment length polymorphism detection (AFLP), polymerase chain reaction (PCR), DNA sequencing, allele specific oligonucleotide (ASO) probes, and hybridization to DNA microarrays or beads. Alternatively, the entire genome could be sequenced. The method may, in certain embodiments, comprise detecting a plurality of polymorphisms in the genome of the plant, for example by obtaining a sample of nucleic acid from a plant and detecting in said nucleic acids a plurality of polymorphisms. The method may further comprise storing the results of the step of detecting the plurality of polymorphisms on a computer readable medium

The disclosure also provides for a food or feed product comprising or consisting of a plant part described herein wherein the plant part can be identified as a part of NUN 06212 SPS. Preferably, the plant part is a spinach leaf or part thereof and/or an extract from a leaf or another plant part described herein. The food or feed product may be fresh or processed, e.g., dried, grinded, powdered, pickled, chopped, cooked, roasted, in a sauce, in a sandwich, pasted, puréed or concentrated, juiced, pickled, canned, steamed, boiled, fried, blanched and/or frozen, etc.

All documents (e.g., patent publications) are herein incorporated by reference in their entirety, including the following cited references:
UPOV, Guidelines for the Conduct of Tests for Distinctness, Uniformity and Stability, TG55/7, world-wide web at upov.int/under edocs/tgdocs/en/tg055.pdf.
US Department of Agriculture, Objective description of Variety Spinach Spinacia oleracea L.)”, world-wide web at ams.usda.gov/under services/plant-variety-protection/pvpo-c-forms under spinach.
Acquaah G., Principles of Plant Genetics and Breeding, 2007, Blackwell Publishing, ISBN-13: 978-1-4051-3646-4
Brotman, Y., et al., Resistance Gene Homologues in Melon are Linked to Genetic Loci Conferring Disease and Pest Resistance, Theor Appl Genet, 2002, vol. 104, pp. 1055-1063.
Colijn-Hooymans, C. M, et. al., Competence for Regeneration of Cucumber Cotyledons is Restricted to Specific Developmental Stages, Plant Cell, Tissue and Organ Culture, 1994, vol. 39, pp. 211-217.
Nikolova V, Niemirowicz-Szczytt Diploidization of cucumber (Cucumis sativus L.) Haploids by Colchicine treatment, Acta Soc Bot Pol 65:311-317
Parvathaneni, Rajiv Krishna, et al., Fingerprinting in Cucumber and Melon (Cucumis spp) Genotypes Using Morphological and ISSR Markers, J. Crop Sci. Biotech, 2011, vol. 14 (1), pp. 39-43.
Ren. Yan et al., Shoot Regeneration and Ploidy Variation in Tissue Culture of Honeydew Melon (Cucumis melo L. inodorus), In Vitro Cell.Dev.Biol.—Plant, 2013, vol. 49, pp. 223-229.
Vos, Pieter, et al., AFLP: A New Technique for DNA Fingerprinting, Nucleic Acids Research, 1995, vol. 23:21, pp. 4407-4414.
US20080222949
US2017/0027126
http://anrcatalog.ucanr.edu/pdf/7216.pdf
https://www.ams.usda.gov/services/plant-variety-protection/pvpo-c-forms
https://www.upov.int/edocs/tgdo cs/en/tg055.pdf
http://cea.cals.cornell.edu/attachments/Cornell CEA baby spinach handbook.pdf

### Examples

### Development of NUN 06212 SPS

The hybrid NUN 06212 SPS was developed from a male and female proprietary inbred line of Nunhems. The female and male parents were crossed to produce hybrid (F1) seeds of NUN 06212 SPS. The seeds of NUN 06212 SPS can be grown to produce hybrid plants and parts thereof (e.g., spinach fruit). The hybrid NUN 06212 SPS can be propagated by seeds or vegetative.

The hybrid variety is uniform and genetically stable. This has been established through evaluation of horticultural characteristics. Several hybrid seed production events resulted in no observable deviation in genetic stability. Coupled with the confirmation of genetic stability of the female and male parents the Applicant has concluded that NUN 06212 SPS is uniform and stable.

### DEPOSIT INFORMATION

A total of 2500 seeds of the hybrid variety NUN 06212 SPS has been deposited according to the Budapest Treaty by Nunhems B. V. on Sep. 24, 2018, at the NCIMB Ltd., Ferguson Building, Craibstone Estate, Bucksburn, Aberdeen AB21 9YA, United Kingdom (NCIMB). The deposit has assigned NCIMB number 43217. A deposit of NUN 06212 SPS and of the male and female parent line is also maintained at Nunhems B. V.

Access to the deposits will be available during the pendency of this application to persons determined by the Director of the U.S. Patent Office to be entitled thereto upon request. Subject to 37 C.F.R. § 1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 U.S.C. § 2321 et seq.).

The most similar variety to NUN 06212 SPS is referred to as NUN 02179 SPS, a variety from Nunhems B. V. with the commercial name Antalia. Table 1 shows a comparison between NUN 06212 SPS and the Reference Variety.

A trial of 40 plants of each variety, from which at least 15 plants or plant parts were randomly selected, are used to measure characteristics. For numerical characteristics, averages are calculated. For non-numerical characteristics, the type/degree are determined. Table 1 lists the UPOV descriptors of NUN 06212 SPS and the Reference Variety.

In one aspect, the disclosure provides a plant having the physiological and morphological characteristics of NUN 06212 SPS as presented in Table 1.

TABLE 1
UPOV Descriptors of Variety NUN
06212 SPS and Reference Variety

| | Application | Reference |
|---|---|---|
| | Variety | Variety |
| UPOV Characteristics | NUN 06212 SPS | Antalia |
| Seedling: length of cotyledon; 3 short/ | 5 | 5 |
| 5 medium/7 long | | |
| Leaf: anthocyanin coloration of | 1 | 1 |
| petioles and veins; 1 absent/9 present | | |
| Leaf blade: intensity of green color; 1 very | 7 | 6 |
| light/2 very light to light/3 light/4 light | | |
| to medium/5 medium/6 medium to dark/7 | | |
| dark/8 dark to very dark/9 very dark | | |
| Leaf blade: blistering; 1 absent or very | 4 | 4 |
| weak/2 very weak to weak/3 weak/4 weak | | |
| to medium/5 medium/6 medium to strong/ | | |
| 7 strong/8 strong to very strong/9 very | | |
| strong | | |
| Leaf blade: lobing; 1 absent or very weak/ | 1 | 5 |
| 3 weak/5 medium/7 strong | | |
| Petiole: attitude; 1 erect/3 semi erect/ | 1 | 3 |
| 5 horizontal | | |
| Petiole: length; 1 very short/3 short/ | 5 | 3 |
| 5 medium/7 strong | | |
| Leaf blade: attitude; 1 erect/3 semi erect/ | 3 | 5 |
| 5 horizontal/7 semi-pendulous | | |
| Leaf blade: shape (excluding basal lobes); | 6 | 6 |
| 1 triangular/2 medium ovate/3 broad | | |
| ovate/4 medium elliptic/5 broad elliptic/ | | |
| 6 circular | | |
| Leaf blade: curving of margin; 1 incurved/ | 2 | 2 |
| 2 flat/3 recurved | | |
| Leaf blade: shape of apex; 1 acute/2 | 3 | 2 |
| obtuse/3 rounded | | |
| Leaf blade: shape in longitudinal section; | 2 | 2 |
| 1 concave/2 flat/3 convex | | |
| Time of start of bolting (for spring sown | 7 | 6 |
| crops, 15% of plants); 1 very early/2 very | | |
| early to early/3 early/4 early to medium/ | | |
| 5 medium/6 medium to late/7 late/8 late | | |
| to very late/9 very late | | |
| Seed: spines (harvest seed); 1 absent/9 | 1 | 1 |
| present | | |

Table 1 contains averaged values. Values may vary due to environment. Other values that are substantially equivalent are also within the scope of the disclosure.

1. A plant, a plant part or a seed of spinach variety NUN 06212 SPS, wherein a representative sample of seed of said variety is deposited under Accession Number NCIMB 43217.

2. The plant part of claim 1, wherein said plant part is a leaf, pollen, an ovule, a fruit, a scion, a root, a rootstock, cutting, flower or a part of any of these or a cell.

3. A seed that produces the plant of claim 1.

4. A seed grown on the plant of claim 1.

5. A spinach plant or a part thereof which does not significantly differ from the plant of claim 1 in any of the following distinguishing characteristics in Table 1: 1) leaf blade intensity of green color; 2) leaf blade blistering; 3) leaf blade lobing; 4) leaf petiole attitude; 5) leaf petiole length; 6) leaf blade attitude; 7) leaf blade shape; and 8) cotyledon length, when grown under the same environmental conditions, and optionally comprises resistance against Peronospora farinose races 7-15 conferred by the RPF11 gene.

6. A spinach plant or a part thereof which does not differ from the plant of claim 1 in any of the characteristics of Table 1, when grown under the same environmental conditions.

7. A tissue or cell culture comprising cells of the plant of claim 1.

8. The tissue or cell culture according to claim 7, comprising cells or protoplasts from said plant part, wherein the plant part is an embryo, meristem, a cotyledon, a hypocotyl, pollen, a leaf, an anther, a root, a root tip, a pistil, a petiole, a flower, a fruit, a seed, a stalk, or a stem.

9. A spinach plant regenerated from the tissue or cell culture of claim 7, wherein the plant has all of the physiological and morphological characteristics of the plant of NUN 06212 SPS, when grown under the same environmental conditions, and wherein a representative sample of seed of NUN 06212 SPS is deposited under Accession Number NCIMB 43217.

10. A method of producing the plant of claim 1, comprising vegetative propagation of at least a part of NUN 06212 SPS, wherein a representative sample of seed of said variety is deposited under Accession Number NCIMB 43217.

11. The method of claim 10, wherein the vegetative propagation comprises regenerating a whole plant from said part of NUN 06212 SPS, wherein a representative sample of seed of said variety is deposited under Accession Number NCIMB 43217.

12. The method of claim 10, wherein the said part is a cutting, a cell culture or a tissue culture.

13. A vegetative propagated plant, or a part of said propagated plant, propagated from the plant or plant part of claim 1, wherein the vegetative propagated plant has all of the physiological and morphological characteristics of the plant of NUN 06212 SPS, when grown under the same conditions, and wherein a representative sample of seed of NUN 06212 SPS is deposited under Accession Number NCIMB 43217.

14. A method of producing a spinach plant, comprising crossing the plant of claim 1 with a second spinach plant at least once, allowing the progeny to form seed and optionally selecting progeny from said crossing.

15. A first-generation progeny plant of the plant of claim 1 obtained by selfing NUN 06212 SPS or cross-pollinating NUN 06212 SPS with another spinach plant.

16. The first-generation progeny plant of claim 15, wherein said progeny plant has all or all but one, two or three of the physiological and morphological characteristics of the plant of NUN 06212 SPS, wherein a representative sample of seed of said variety is deposited under Accession Number NCIMB 43217, when grown under the same environmental conditions.

17. A spinach plant having one, two or three physiological and morphological characteristics which are different from those of the plant of claim 1, and which otherwise has all the physiological and morphological characteristics of NUN 06212 SPS, when grown under the same environmental conditions.

18. The plant of claim 17, where the different characteristic is effected by a mutation or by transformation.

19. The plant of claim 1, further comprising a single locus conversion, wherein said plant has all or all but one, two or three of the morphological and physiological characteristics of the plant of NUN 06212 SPS, wherein a representative sample of seed of said variety is deposited under Accession Number NCIMB 43217, when grown under the same environmental conditions, optionally wherein the single locus conversion confers a trait of male sterility, herbicide tolerance, insect resistance, pest resistance, disease resistance, environmental stress tolerance, Peronospora resistance, modified carbohydrate metabolism or modified protein metabolism.

20. A doubled haploid cell or plant made from haploid cells of NUN 06212 SPS, wherein a representative sample of seed of NUN 06212 SPS is deposited under Accession Number NCIMB 43217.

21. A container comprising a plant, a plant part or a seed of claim 1.

22. A food or feed product or a processed product comprising the plant part of claim 2.

23. A method of producing a spinach leaf, comprising growing the plant of claim 1 until it sets at least one leaf, and collecting the leaf.

24. A method for inducing mutation in the plant of claim 1, comprising
a. exposing a seed, a plant or a plant part of NUN 06212 SPS to a mutagenic chemical or to radiation, wherein a representative sample of seed of NUN 06212 SPS is deposited under Accession Number NCIMB 43217; and
b. selecting a seed, a plant or a plant part or a cell of NUN 06212 SPS having a mutation.

25. A method collecting pollen of NUN 06212 SPS comprising growing a plant of claim 1 until at least one flower contains pollen and collecting the pollen.

26. A method of producing a spinach plant having a desired trait, wherein the method comprises mutating a plant of variety NUN 06212 SPS and selecting a plant with a desired trait, wherein the mutated plant contains the desired trait and retains all or all but one of phenotypic and morphological characteristics of variety NUN 06212 SPS, and wherein a representative sample of seed of NUN 06212 SPS is deposited under Accession Number NCIMB 43217, when grown under the same environmental conditions.

27. A spinach plant comprising at least a first set of the chromosomes of the plant part of claim 1, wherein a representative sample of said seed variety has been deposited under Accession Number NCIMB 43217, and wherein said spinach plant comprising said first set of the chromosomes has all the physiological and morphological characteristics of NUN 06212 SPS.
